(19) United States
(12) Patent Application Publication
(10) Pub. No.: US 2024/0002903 A1
(43) Pub. Date: Jan. 4, 2024

(54) METHOD FOR AMPLIFYING FREE NUCLEIC ACIDS DIRECTLY FROM ONE DROP OF UNPURIFIED SAMPLE

(71) Applicant: Kuo Ping CHIU, New Taipei City (TW)

(72) Inventor: Kuo Ping CHIU, New Taipei City (TW)

(21) Appl. No.: 17/940,975

(22) Filed: Sep. 8, 2022

(30) Foreign Application Priority Data
Jul. 1, 2022 (TW) 111124736

(51) Int. Cl. C12Q 1/6806; C12Q 1/686

(52) U.S. Cl. CPC C12Q 1/6806; C12Q 1/686; C12Q 2600/16; C12Q 2523/113; C12Q 2523/109; C12Q 2531/113

(57) ABSTRACT

A method for amplifying free nucleic acids (NAs) directly from one drop unpurified crude sample. The sample comprises nucleic acid fragments. The method includes (a) mixing an unpurified sample with a buffer to form an uniformly mixed solution; (b) adding the mixed solution to a tube then heating the tube to denature the proteins in the mixed solution, and cooling to room temperature; (c) subjecting he nucleic acid fragments in the mixture to a processing reaction required for adaptor-dependent PCR; (d) performing a ligation reaction between the processed nucleic acid fragments and adapters, forming nucleic acid fragments ligated with adapter in both ends, wherein the adapter is a complementary double-stranded nucleic acid (dsNA) fragment, one of which is an oligonucleotide with 5′-phosphate and the other is an oligonucleotide with thymine (T) or uracil (U); and (e) performing the adapter-dependent PCR to the nucleic acid fragments in the mixed solution.

### BACKGROUND OF THE INVENTION

### 1. Technical Field

Ligation of adapters to the termini of the unknown nucleic acid (NA) target molecules is essential for PCR amplification of the unknown NA molecules, while such adapter-dependent PCR amplification is in turn essential for next-generation sequencing (NGS) of the unknown NA fragments. The present invention relates to a method for direct adapter ligation and PCR amplification of free nucleic acids in a mixture containing a drop of crude body fluid sample without pre-purification of the nucleic acid molecules. The invention can amplify nucleic acids in a simple manner to facilitate the analysis of the nucleic acids in the sample.

### 2. Description of Related Art

Polymerase chain reaction (PCR) is a method by which the nucleic acids in a sample can be precisely amplified, and the amplified nucleic acid molecules can be subjected to sequencing or other tests in order to detect, for example, disease-related gene mutations, gene copy number variation (CNV), genomic alterations in cells (e.g., chromosomal abnormality), or pathogens (e.g., pathogenic bacteria or viruses). Thus, PCR has ample applications in diagnosis.

Current PCR amplification of minute amount of unknown nucleic acids that may present in a body fluid is severely hindered by two technical obstacles. First, the nucleic acids in the sample must be purified first by a series of complicated steps that normally entail commercial spin column or magnetic beads, and the sample can then be subjected to end-modification and adapter ligation of the nucleic acid molecules, which are essential for PCR amplification. Second, as an attempt to increase the quantity of NAs, several milliliters of body fluid are generally required. This is because the NAs in body fluids are normally of low abundance and the requirement of pre-isolation of NAs further reduces the quantity during the purification process.

The aforesaid sampling and purification processes not only incur extra cost and waste more time, the pre-isolation also inevitably causes substantial loss (normally >20%) of the precious nucleic acid molecules during the process. Besides, pre-isolation also introduces bias into the analysis because small NA molecules are more likely to be lost and different isolation kits may have different sequence preference. Taken together, these problems not only increase the difficulty of material manipulation, but also introduce significant uncertainty and bias into the analysis. Thus, the requirement of pre-isolation of NAs from a liquid sample is not suitable for the amplification and analysis, either by sequencing or other methods, of minute NAs, especially for the cell-free nucleic acids (cfNAs), forensic DNA samples, or degraded nucleic acids in fossil specimen.

### BRIEF SUMMARY OF THE INVENTION

In view of the above, the inventor of the present invention developed a novel and simple method allowing adapter-dependent PCR to amplify nucleic acids directly from crude sample without pre-PCR isolation (i.e., pre-isolation, in short) of the nucleic acids, so that the amplification can be simplified and expedited and the quality of analysis can be significantly improved. This method relies on the ultra-high ligation efficiency of the adapter, which can occur even under the interference by various types of proteins and other molecules in the reaction environment.

In particular, an main aspect of the present invention is to provide a method for amplifying free nucleic acids directly from a crude sample, wherein the unpurified sample comprises nucleic acid fragments, the method comprising the sequential steps of: (a) dilution: mixing the unpurified sample with a buffer thoroughly to form a mixed solution; (b) protein denaturation: adding the mixed solution into a test tube, heating the mixed solution in the test tube to denature proteins, and then cooling the mixed solution to room temperature; (c) end modification of nucleic acid molecules: subjecting the nucleic acid fragments in the mixed solution to a processing reaction required for an adapter-dependent polymerase chain reaction (PCR); (d) adapter ligation: performing a ligation reaction between the processed nucleic acid fragments and double-stranded adapters having a single type of sequence, defined as double-stranded homogeneous adapters, in order to form nucleic acid fragments each ligated at two ends thereof with one said double-stranded homogeneous adapter, wherein each said double-stranded homogeneous adapter is a complementary double-stranded nucleic acid (dsNA) fragment with one strand being an oligonucleotide carrying a 5′-phosphate group and the other strand being an oligonucleotide carrying a thymine (T) or uracil (U); and (e) PCR: adding components required for performing the adapter-dependent PCR on the ligated nucleic acid fragments, and then performing the adapter-dependent PCR on the ligated nucleic acid fragments in the mixed solution.

According to one or more examples of the present invention, the nucleic acid fragments in the unpurified sample are dsNA fragments.

According to one or more examples of the present invention, the unpurified sample is in an amount of 0.1 μL to 100 μL.

According to one or more examples of the present invention, the steps (b) to (e) are carried out in the same test tube.

According to one or more examples of the present invention, the test tube is provided therein with a filter, and the step (b) comprises adding the mixed solution onto the filter in order for the mixed solution to pass through the filter and then flow to the bottom of the test tube.

According to one or more examples of the present invention, the processing reaction in the step (c) include 1) an end modification performed on the nucleic acid fragments in the mixed solution in order for the nucleic acid fragments to form nucleic acid fragments each having a blunt end, wherein the blunt ends are formed by repairing the nucleic acid fragments in the mixed solution or cleaving the nucleic acid fragments in the mixed solution with an enzyme and 2) a 3′-A-tailing reaction performed on the nucleic acid fragments in the mixed solution in order for the nucleic acid fragments to form nucleic acid fragments each having a 3′-A sticky ends.

According to one or more examples of the present invention, the adapter-dependent PCR in the step (e) is performed by using the nucleic acid fragments ligated with the double-stranded homogeneous adapters as templates, and by using a single type of bidirectional primer corresponding to the double-stranded homogeneous adapters.

According to one or more examples of the present invention, the adapter-dependent PCR is a digital PCR (dPCR).

According to one or more examples of the present invention, the method further comprises the step (f) of purifying a product of the adapter-dependent PCR.

According to one or more examples of the present invention, the method further comprises the step (g) of analyzing the product of the adapter-dependent PCR by a sequencing or diagnostic method.

The present invention is advantageous over the conventional PCR techniques in that the invention requires only one drop, or even a minute amount, of sample and can amplify the nucleic acids in the sample in a simple manner, thereby overcoming the problem that the cfNA (including cell-free DNA (cfDNA) and cell-free RNA (cfRNA)) in a sample tends to exist in a small amount and tend to be lost during purification. The invention can amplify specific gene fragments of different sizes in a nucleic acid sample to facilitate subsequent analysis, to reduce the expenses and operation time of experiments, to increase the sensitivity and accuracy of nucleic acid analysis, and to thereby resolve the aforesaid difficulty in, and remove the aforesaid limitation on, nucleic acid amplification and analysis, namely the unsuitability of the existing PCR techniques for amplifying and analyzing the free DNA and RNA in a body fluid, the nucleic acids in a minute amount of body fluid sample, and the minute amount of nucleic acids in other samples.

### BRIEF DESCRIPTION OF THE SEVERAL VIEWS OF THE DRAWINGS

In order to better understand the technical content of the present invention, the accompanying drawings show preferred embodiments of the present invention. However, it should be understood that the present invention is not limited to the technical contents shown in the accompanying drawings.

FIG. 1 shows the test tube that can be used in the method of the present invention;

FIG. 2 shows a comparison of the experimental process flows of the method of the invention and of the ThruPLEX method;

FIG. 3A shows a profile of original, or unamplified, plasma cfDNA, FIG. 3B shows a profile of plasma cfDNA that has been amplified by the method of the invention, and FIG. 3C shows a profile of plasma cfDNA that has been amplified by a commercially available ThruPLEX kit;

FIG. 4A shows a profile of original, or unamplified, saliva cfDNA, FIG. 4B shows a profile of saliva cfDNA that has been amplified by the method of the invention, and FIG. 4C shows a profile of saliva cfDNA that has been amplified by a commercially available ThruPLEX kit; and

FIG. 5A shows a profile of original, or unamplified, urinary cfDNA, FIG. shows a profile of urinary cfDNA that has been amplified by the method of the invention, and FIG. 5C shows a profile of urinary cfDNA that has been amplified by a commercially available ThruPLEX kit.

### DETAILED DESCRIPTION OF THE INVENTION

The following provides a detailed description on the embodiments of the present invention. However, such description and the embodiments provided shall not be used to limit the scope of the present invention. Any modification and change made by a person with ordinary skills in the technical field of the present invention based on the embodiments disclosed by the present invention and within the principle and scope of the present invention shall be treated to be within the scope of the present invention.

The term “one” or “a” described in the following content shall mean one or more than one, i.e., at least one.

The term “comprising, having or including” described in the following content shall mean the existence of one or more than one parts, steps, operations and/or elements or the inclusion of such parts, steps, operations and/or elements.

The term “approximately or about” or “basically” described in the following content shall mean that a certain value or range is close to an acceptable specified tolerance, and the purpose of the use of such term is to prevent a third party's unreasonable, illegal or unfair interpretation of a value or range disclosed by the present invention to be within or equivalent to the exact or absolute value or range disclosed by the present invention only.

The primary objective of the present invention is to provide a method for amplifying free nucleic acids directly from one drop of unpurified sample that includes nucleic acid fragments. The method includes the following steps: (a) dilution: mixing the unpurified sample with a buffer thoroughly to form a mixed solution; (b) protein denaturation: adding the mixed solution into a test tube, heating the mixed solution in the test tube to denature the proteins in the mixed solution, and then cooling the mixed solution to room temperature; (c) end modification of nucleic acid molecules: subjecting the nucleic acid fragments in the mixed solution to a processing reaction required for adapter-dependent PCR; (d) adapter ligation: performing a ligation reaction between the processed nucleic acid fragments and double-stranded adapters having a single type of sequence (defined herein as double-stranded homogeneous adapters), in order to form nucleic acid fragments each ligated with two double-stranded homogeneous adapters at the two ends respectively, wherein each double-stranded homogeneous adapter is a complementary double-stranded nucleic acid (dsNA) fragment with one strand being an oligonucleotide carrying a 5′-phosphate group and the other strand being an oligonucleotide carrying a thymine (T) or uracil (U); and (e) PCR: adding the components required for performing the adapter-dependent PCR on the ligated nucleic acid fragments, and then performing the adapter-dependent PCR on the ligated nucleic acid fragments in the mixed solution.

As used herein, the term “sample” refers to a nucleic-acid-fragment-containing body fluid sample of an organism, a nucleic-acid-fragment-containing tissue sample for forensic use, or any other sample in which the nucleic acid fragments are to be detected and amplified, such as but not limited to a fossil sample; the present invention has no limitation in this regard. The nucleic acid fragments in the sample are preferably, but not limited to, dsNA fragments, and the dsNA fragments in the invention preferably come from a body fluid sample of an organism, such as but not limited to blood, blood plasma, saliva, urine, tear, cerebrospinal fluid, or other fluids secreted by an organism; the invention has no limitation in this regard. The organism may be a mammal or a non-mammalian animal, wherein the mammal may be, for example but not limited to, a human, a non-human primate, sheep, a dog, a member of the Glade Glires (e.g., a mouse or rat), a guinea pig, a cat, a rabbit, an ox, or a horse, and wherein the non-mammalian animal by be, for example but not limited to, a rooster, an amphibian, or a reptile. The organism in the invention is preferably a human. In addition, the dsNA fragments in the sample are preferably cfNA, such as cfDNA or cfRNA.

As used herein, the term “unpurified sample” refers to a sample that has not been purified by a conventional nucleic acid purification method (e.g., extraction and purification with a spin column or with magnetic beads), and whose original form is a liquid or can be rendered into a liquid. The term “unpurified sample” as used herein may also refer to a sample that has been centrifuged or has been processed by another method. For example, if the original form of an unpurified sample is not a liquid or cannot be rendered into a liquid (e.g., a tissue embedded in paraffin, or a fossil), the unpurified solid sample must be pretreated, i.e., rendered into a liquid or rendered liquefiable, in order to serve as the sample in the nucleic acid amplification method of the present invention. If the sample is a tissue embedded in paraffin, the pretreatment may be, for example, a dewaxing procedure; the invention has no limitation in this regard. If the sample is a fossil, the pretreatment may be, for example, pulverizing the fossil in advance; the invention has no limitation in this regard, either.

When performing a conventional PCR technique, a relatively large amount (at least several c.c.) of sample is required because the nucleic acid fragments in the sample tend to be few, are subject to loss, and have to be purified before the PCR. By contrast, the inventor of the present invention has found through repeated experiments that the method of the invention can amplify the nucleic acids in a sample as small as one drop (i.e., about 10 μL), without any purification steps performed in advance; that is to say, the method of the invention enables amplification and analysis of the minute amount of nucleic acid fragments in a sample. In step (a), therefore, the amount of the unpurified sample may be 0.1 to 50 μL, such as but not limited to an amount ranging between any two of the following: 0.1 μL, 0.5 μL, 1 μL, 5 μL, 10 μL, 15 μL, 20 μL, 25 μL, 30 μL, 35 μL, 40 μL, 45 μL, 50 μL and 100 μL. In the invention, the amount of the unpurified sample preferably ranges from about 10 μL to about 20 μL.

The “buffer” used in step (a) refers to a liquid that can be used to stabilize the pH value and that facilitates the release of dsNA fragments, such as but not limited to water, a Tris-EDTA (TE) buffer, or a phosphate-buffered saline (PBS).

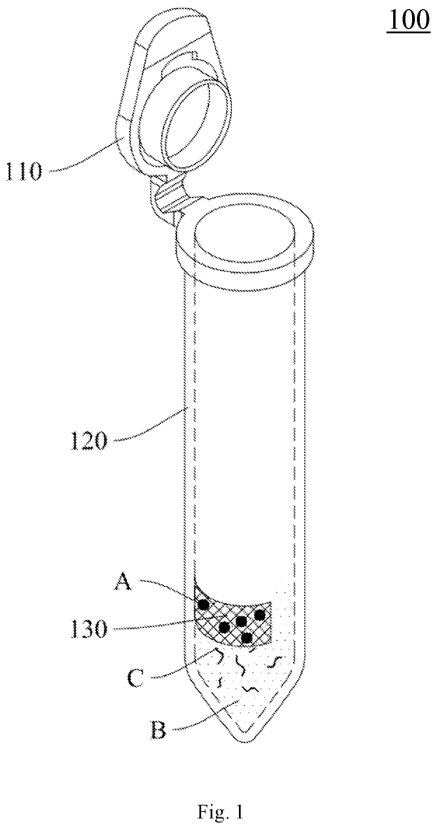

Please refer to FIG. 1 for the test tube used in the method of the present invention.

The “test tube” used in step (b) preferably refers to a test tube 100 that includes a filter 130. The test tube 100 is preferably composed of a cover 110 and a tube body 120 provided under the cover 110, with the filter 130 provided on the inner tube wall of the tube body 120. When a mixed solution B formed by mixing the unpurified sample or pretreated sample with the buffer thoroughly is added to the filter 130, which is made by interweaving multiple layers of fibrous elements and therefore has a plurality of pores, impurities A that may exist in the mixed solution B (e.g., cells, cell membranes, proteins, intercellular substances, and cell fragments) will get stuck in the pores and thus be prevented from flowing to the bottom of the test tube 100 along with the liquid in the sample. By contrast, dsNA fragments C, which are relatively small and slender, are allowed to flow to the bottom of the test tube 100 along with the liquid in the sample.

In order to make effective use of the dsNA fragments C in the sample, the mixed solution B in a preferred embodiment is in such an amount that the filter 130 can contact the mixed solution B and thus be kept moist during the subsequent PCR process, allowing the small amount of dsNA fragments C that did not flow to the bottom of the test tube 100 along with the liquid in the sample but remain attached to the filter 130 to be able to undergo various reactions while staying on the filter 130. Moreover, the amount of the mixed solution B can be adjusted during or after step (b); that is to say, there is no need to add more mixed solution B unless the current amount of the mixed solution B is found to be unable to keep the filter 130 moist. Therefore, while the present invention omits the advance nucleic acid purification procedure of the conventional PCR techniques, the filter 130 in the test tube 100 is directly used to retain substances that may affect various reactions (such as but not limited to PCR), i.e., to make those substances stuck in the pores of the filter 130, so that the reactions involved in subsequent nucleic acid amplification (e.g., steps (c) to (e) of the invention) can be carried out directly in the same test tube.

In one or more embodiments, the area of the filter 130 may be greater than, equal to, or less than the greatest cross sectional area of the tube body 120, whether the filter 130 is disposed vertically or slantingly with respect to the tube body 120 (the present invention has no limitation on the slant angle of the filter 130, provided that the mixed solution B can be added to the filter 130 in drops). Furthermore, the filter 130 may have a circular or rectangular shape; the invention has no limitation in this regard. The invention has no limitation on the thickness of the filter 130, either, provided that the filter 130 can effectively make the impurities A in the mixed solution B stuck in the filter 130 and allow only the dsNA-fragment-C-containing portion of the mixed solution B to flow to the bottom of the test tube 100. In one preferred embodiment, the filter 130 can be put directly into the tube body 120 without being fixed therein, and when the mixed solution B is added into the tube body 120, the filter 130, which is porous and therefore has relatively low density, will float in the tube body 120.

In one or more embodiments, the material of the filter 130 may be, for example but not limited to, cellulose, nitrocellulose, cellulose acetate, polyvinylidene fluoride (PVDF), glass fiber, nylon, or polyketone; the present invention has no limitation in this regard. The filter 130 in the invention, therefore, may be made of paper, cloth, or other materials that have a filtering property.

In one or more embodiments, the pores of the filter 130 have diameters ranging from 0.2 to 20 μm, such as but not limited to 0.2-20 μm, 0.2-15 μm, 0.2-10 μm, 0.2-5 μm, 0.5-20 μm, 0.5-15 μm, 0.5-10 μm, or 0.5-5 μm.

In one or more embodiments, the tube body 120 may have, for example but not limited to, the conical shape shown in FIG. 1 and may be cylindrical instead; the present invention has no limitation in this regard.

In addition, in order to denature the proteins in the mixed solution B and thereby make the proteins stuck in the pores of the filter 130 without flowing to the bottom of the tube body 120 along with the mixed solution B, and hence without affecting the subsequent PCR, the heating temperature used in step (b) to denature the proteins in the mixed solution may range from 50 to 80° C., such as but not limited to a temperature ranging between any two of the following: 50° C., 55° C., 60° C., 65° C., 70° C., 75° C., and 80° C., and the heating time may range from 5 to 30 minutes, such as but not limited to 5 minutes, 10 minutes, 15 minutes, 20 minutes, 25 minutes, or 30 minutes. A suitable heating temperature and heating time can be determined according to the type of the sample.

The inventor of the present invention has found that if the sample contains a relatively small amount of impurities A, the test tube used in step (b) may be a conventional test tube, i.e., a test tube without the filter structure; in other words, the test tube used in step (b) may dispense with the filter 130 in FIG. 1 when the sample need not be filtered.

The “processing reaction” in step (c) refers to an advance processing reaction that allows the nucleic acid fragments in the mixed solution to ligate with the double-stranded homogeneous adapters (i.e., double-stranded adapters having a single type of sequence) so that the nucleic acid fragments can take part in the subsequent adapter-dependent PCR. More specifically, the processing reaction in the present invention refers to either performing an A-tailing (also known as dA-tailing) reaction on the 3′ ends of the nucleic acid fragments in the mixed solution in order for the nucleic acid fragments to form nucleic acid fragments with a 3′-A overhang (sticky end), or performing end modification on the nucleic acid fragments in the mixed solution in order to trim the ends of the nucleic acid fragments in the mixed solution, thereby forming nucleic acid fragments with a blunt end.

In one or more embodiments, the 3′-A-tailing reaction can be carried out using a conventional method or kit, such as the NEBNext® Ultra End Repair/dA-Tailing Module (NEB, E7442S/L); the present invention has no limitation in this regard. In one or more embodiments, the end modification method used to form blunt ends may be repairing the nucleic acid fragments in the mixed solution or cleaving the nucleic acid fragments in the mixed solution with an enzyme. If enzymatic cleavage is used, the enzyme may be a restriction enzyme such as but not limited to PovII, SmaI, HaeIII, AluI, or EcoRV; the invention has no limitation in this regard.

The “double-stranded homogeneous adapters”, or “double-stranded adapters having a single type of sequence”, used in step (d) refers to complementary dsNA fragments having a single type of sequence, with the two strands of each complementary dsNA fragment being respectively an oligonucleotide carrying a 5′-phosphate group and an oligonucleotide carrying a thymine (T) or uracil (U), wherein the thymine (T) or uracil (U) may be an overhang (sticky end) or a blunt end, and wherein the complementary dsNA fragments do not self-ligate. The term “adapter” indicates that the double-stranded homogeneous adapters can ligate with the oligonucleotides at the ends of the dsNA molecules in the sample. The double-stranded homogeneous adapters may have lengths ranging from 10 to 50 bases, preferably from 10 to 30 bases, more preferably from 10 to 20 bases. A length less than 10 nucleotides may lower the specificity of annealing, and a length greater than 20 nucleotides may render an experiment uneconomical.

The “adapter-dependent PCR” in step (e) refers to an adapter-dependent PCR in which nucleic acid fragments ligated with the double-stranded homogeneous adapters are used as templates, and in which a single type of bidirectional primer corresponding to the double-stranded homogeneous adapters is used. The term “a single type of bidirectional primer” refers to a constituent oligonucleotide of the double-stranded homogeneous adapters. Therefore, as long as the dsNA fragments in the sample have the double-stranded homogeneous adapters (i.e., double-stranded adapters having a single type of sequence), the single-type oligonucleotide can be used as a reverse primer as well as a forward primer (hence the term “bidirectional”) to guide the polymerase in annealing the forward strand and reverse strand in the elongation step of the PCR.

In one or more embodiments, the adapter-dependent PCR may be, for example but not limited to, a touchdown PCR, a reverse transcription PCR (RT-PCR), a hot start PCR, a real-time PCR/quantitative PCR, a nested PCR, a multiplex PCR, a reconditioning PCR, a co-amplification at lower denaturation temperature PCR (COLD-PCR), or a digital PCR (dPCR). The adapter-dependent PCR in the present invention is preferably a digital PCR.

In one or more embodiments, step (e) may be followed by: step (f) of purifying the product of the adapter-dependent PCR; and step (g) of analyzing the product of the adapter-dependent PCR by a sequencing or diagnostic method. The product of the adapter-dependent PCR may be purified by a conventional method; the present invention has no limitation in this regard. The sequencing or diagnostic method may be, for example but not limited to, Sanger sequencing, next-generation sequencing (NGS) (e.g., Illumina), single-molecule sequencing (e.g., nanopore or PacBio), or ion torrent sequencing; the invention has no limitation in this regard, either.

An embodiment of the present invention and a comparative example are described below in order for a person of ordinary skill in the art to understand the essential technical contents of the invention with ease. As the invention can be changed and/or modified in many ways without departing from the spirit or scope of the invention, all such changes and modifications made to adapt to different uses or conditions shall fall within the scope of the appended claims.

### Embodiment

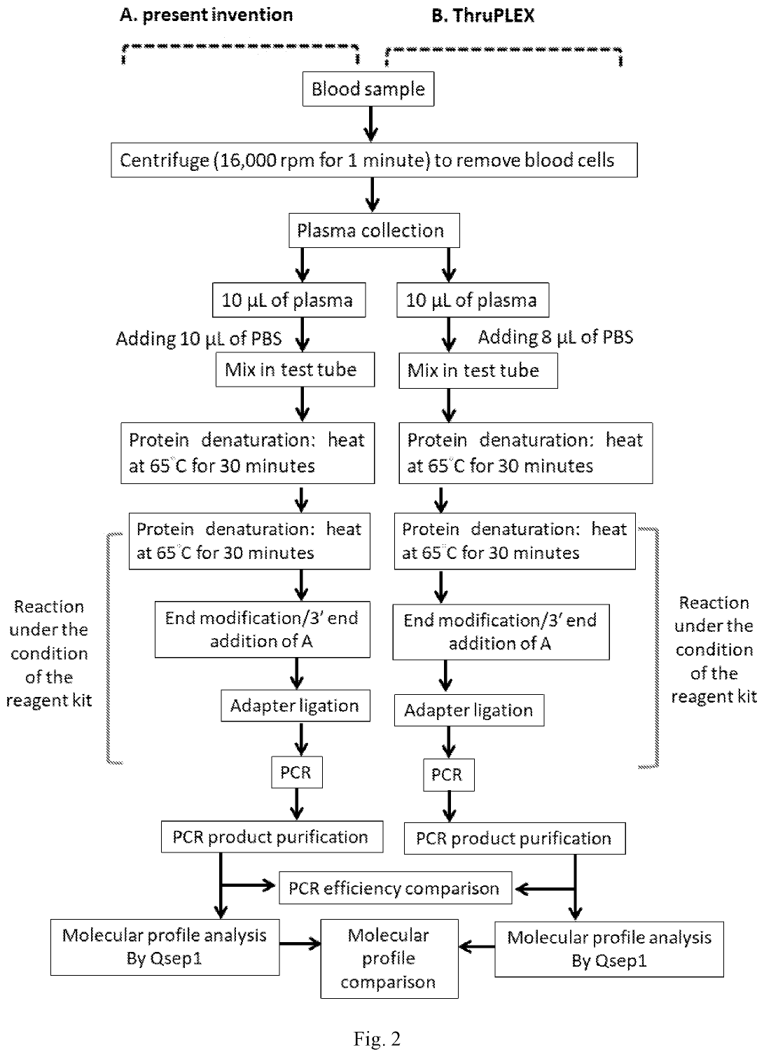

Please refer to FIG. 2 for a comparison of the experimental process flows of the method of the present invention and of the ThruPLEX method.

[Dilution]

An appropriate amount of blood was collected from a test subject. The blood was centrifuged at 16,000 rpm for 1 minute to remove the blood cells and thereby obtain a plasma sample. 10 μL of the plasma sample and 10 μL of 1× PBS were thoroughly mixed in a test tube to form 20 μL of mixed solution.

[Protein Denaturation]

The mixed solution, which contained the plasma sample, was heated at 65° C. for 30 minutes to denature the proteins in the mixed solution, lest the proteins interfere with the subsequent nucleic acid amplification reaction.

[End Modification of Nucleic Acid Molecules]

An end modification buffer and an end modification enzyme were added into the test tube and sufficiently mixed, before the test tube was placed in a PCR heating block. The contents of the test tube were first heated at 20° C. for 30 minutes, then heated at 65° C. for 30 minutes, and then allowed to rest at 4° C. in order to be cooled.

[Adapter Ligation]

The mixed solution in the test tube (which solution now contained end-modified cfDNA fragments) was added with a ligation buffer containing double-stranded homogeneous adapters (i.e., the “adapter ligation” step in FIG. 2) and a 2× ligation mixture. After a sufficient mix, the mixed solution was heated at 20° C. for 4 hours in order for the ligase in the ligation mixture to ligate the double-stranded homogeneous adapters to the cfDNA fragments, thereby producing cfDNA fragments ligated with the double-stranded homogeneous adapters, wherein the 3′-T or 3′-U overhang of each double-stranded homogeneous adapter was complementarily ligated to the 3′-A overhang of a cfDNA fragment. The double-stranded homogeneous adapters were each formed by annealing two complementary single-stranded nucleic acid fragments, one of which was an oligonucleotide carrying a 5′-phosphate group, and the other of which was an oligonucleotide carrying a 3′-T or 3′-U, with the 3′-T or 3′-U being an overhang. The double-stranded homogeneous adapters were designed according to Published Taiwan Invention Patent Application No. 202035699A, which is incorporated herein by reference.

[PCR]

The test tube containing the plasma sample was centrifuged briefly, before the supernatant was collected and transferred into another test tube. A T3 oligonucleotide and other components required for adapter-dependent PCR were then added into the supernatant-containing test tube to form a mixed solution for reaction.

The mixed solution for reaction was subjected to initial denaturation at 98° C. for 30 seconds, followed by 20 adapter-dependent PCR cycles, with the final elongation step performed at 72° C. for 5 minutes. Once the adapter-dependent PCR was terminated by adding an exonuclease, an adapter-dependent PCR product was obtained.

[Purification of the PCR Product]

The PCR product was purified with magnetic beads and then eluted with nuclease-free water or a 0.1× TE buffer to obtain 30 μL of purified PCR product, which was kept at −20° C.

[Molecular Profile Analysis]

The magnetic-bead-purified PCR product was applied to QSep1 in order to obtain the molecular profile of the product.

### Comparative Example

The comparative example used the same experimental method as the foregoing embodiment. An appropriate amount of plasma was collected from the test subject. 10 μL of the plasma sample and 8 μL of 1× PBS were thoroughly mixed in a test tube to form 18 μL of mixed solution. After denaturing the proteins in the mixed solution, the cfDNA in the unpurified sample was directly amplified according to the user manual of the ThruPLEX Tag-Seq Kit (Clontech, catR400584). The steps performed are detailed in FIG. 2.

Aside from the plasma sample, appropriate amounts of saliva and urine were also collected from the test subject, and a saliva sample and a urine sample were prepared and analyzed in accordance with the experimental process flows shown in FIG. 2 of the method of the present invention (as part of the foregoing embodiment) and of the ThruPLEX method (as part of the foregoing comparative example).

[Experimental Results]

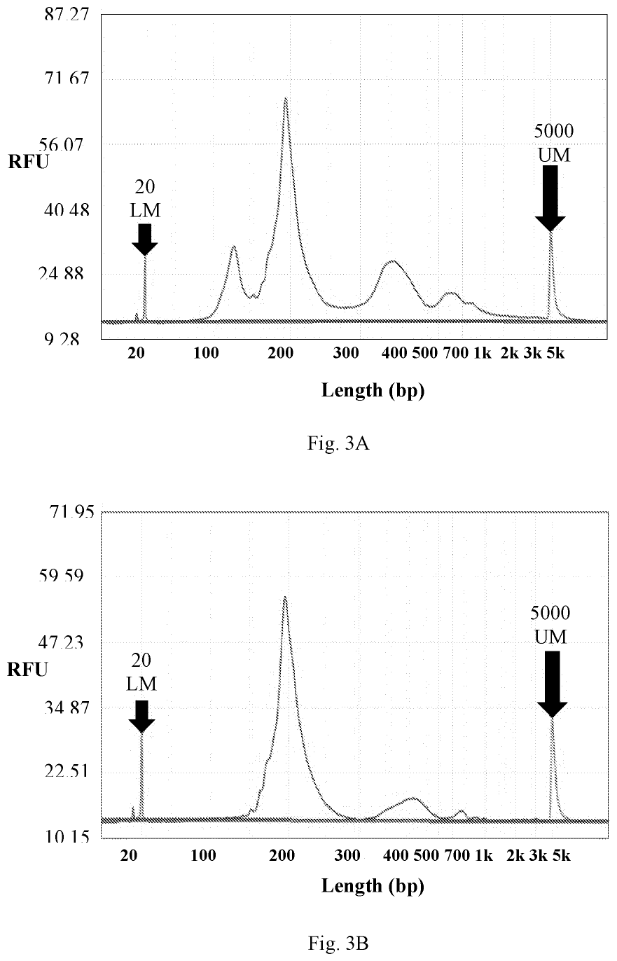

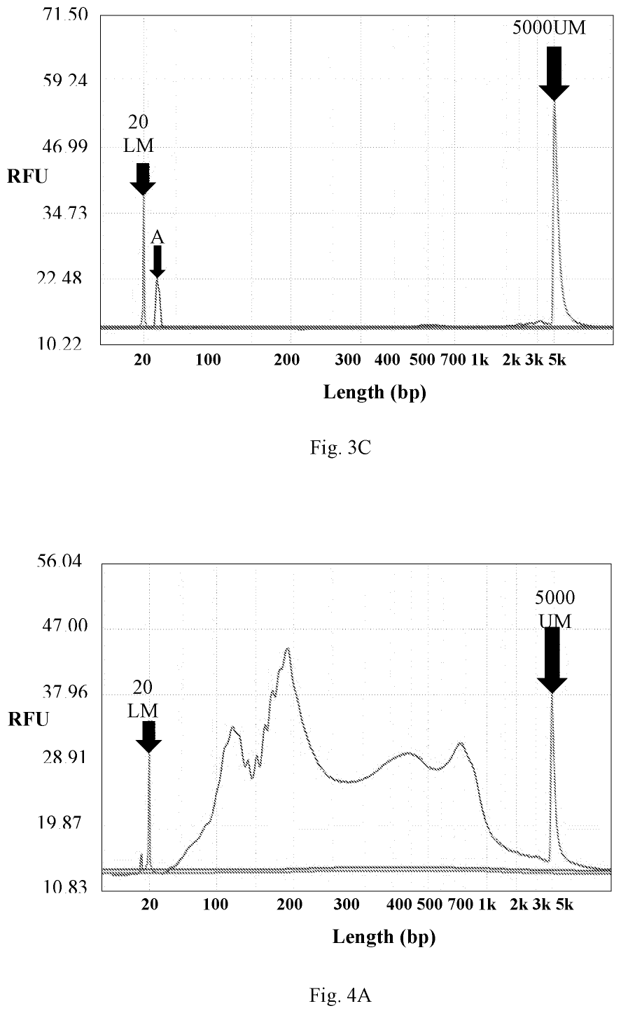

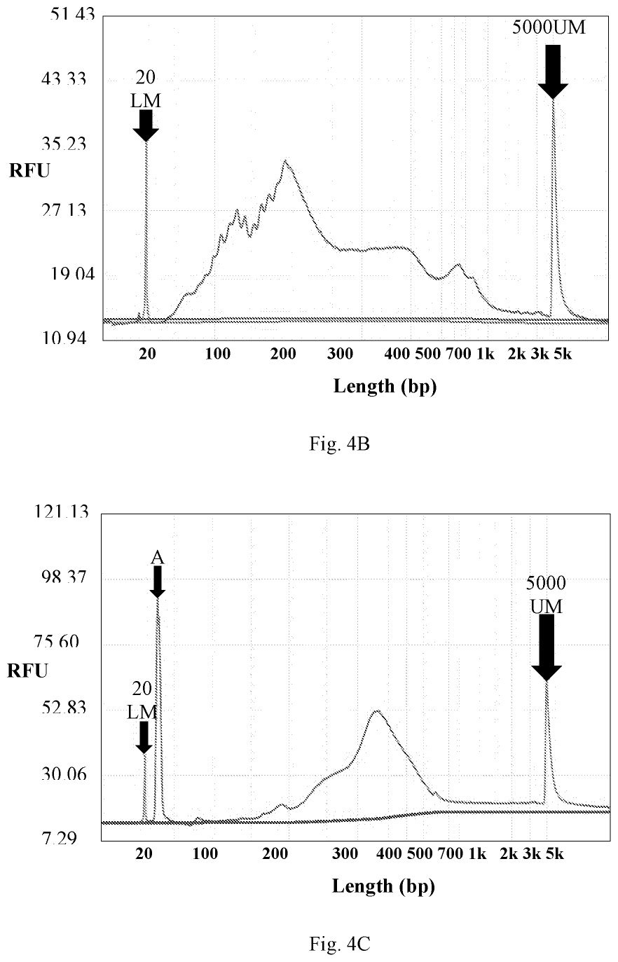

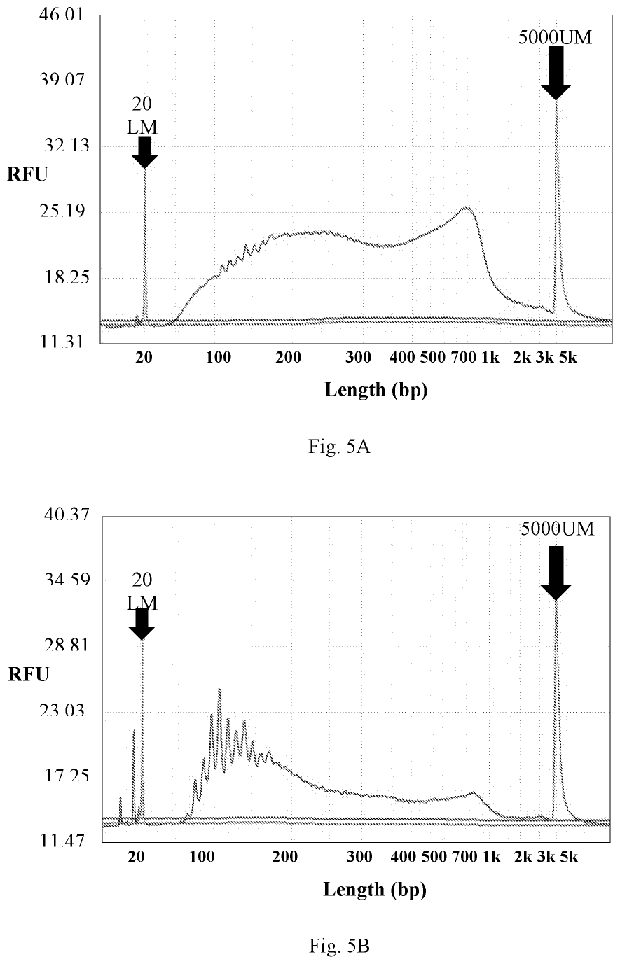

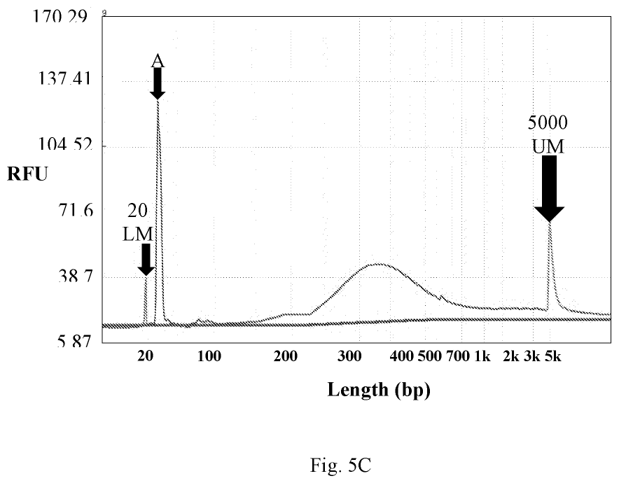

Please refer to the experimental results in FIG. 3, FIG. 4, and FIG. 5, which show the profiles obtained after amplifying the cfDNA in the plasma samples, the saliva samples, and the urine samples with the method of the present invention (as part of the foregoing embodiment) and the commercially available ThruPLEX Tag-Seq Kit (as part of the foregoing comparative example).

FIG. 3A shows the experimental result of the plasma control, or more particularly the profile of 150 ng of cfDNA in a purified yet unamplified plasma sample. FIG. 3B shows the profile of 150 ng of cfDNA taken from PCR products generated from 10 μL of unpurified plasma sample by PCR amplification performed according to the method of the present invention. FIG. 3C shows the profile of 75 ng of cfDNA (note: the final product obtained was less than 150 ng, so only 75 ng was used) taken from PCR products generated from 10 μL of unpurified plasma sample by PCR amplification performed according to the ThruPLEX method. The peaks (indicated by arrows as 20 LM and 5000 UM, respectively) at the two end points of each profile in FIG. 3A, FIG. 3B, and FIG. 3C represent 20 bp and 5000 bp size markers, respectively.

Compared with the original profile in FIG. 3A, the profile in FIG. 3B, which was obtained after amplification according to the method of the present invention, shows with precision the shape of the original profile of the cfDNA in the control plasma sample. More specifically, the data peaks at 200 bp and around 400 bp in the control group can be seen respectively at 200 bp and around 400 bp in the result corresponding to the method of the invention. By contrast, the profile in FIG. 3C, which corresponds to the ThruPLEX method, not only fails to show the shape of the original cfDNA profile precisely (meaning the ThruPLEX method failed to amplify the cfDNA in the plasma sample with precision), but also shows an artifact at about 34 bp (as indicated by the s arrow A in FIG. 3C). It is obvious that the cfDNA amplified by the ThruPLEX method does not show the profile of the original cfDNA precisely. Therefore, the experimental results of the plasma samples have proved that, without having to perform such conventional PCR steps as purification with a spin column or magnetic beads before the PCR, the method of the invention can produce satisfactory nucleic acid amplification results that are unachievable with the commercially available DNA amplification kit.

FIG. 4A shows the experimental result of the saliva control group, or more particularly the profile of 150 ng of cfDNA in a purified yet unamplified saliva sample. FIG. 4B shows the profile of 150 ng of cfDNA taken from PCR products generated from 10 μL of unpurified saliva sample by PCR amplification performed according to the method of the present invention. FIG. 4C shows the profile of 150 ng of cfDNA taken from PCR products generated from 10 μL of unpurified saliva sample by PCR amplification performed according to the ThruPLEX method. The data peaks (indicated by the arrows as 20 LM and 5000 UM respectively) at the two end points of each profile in FIG. 4A, FIG. 4B, and FIG. 4C represent 20 bp and 5000 bp size markers, respectively.

Compared with the original profile in FIG. 4A, the profile in FIG. 4B, which was obtained after amplification according to the method of the present invention, shows with precision the shape of the original profile of the cfDNA in the control saliva sample. More specifically, the data peaks around 100 bp, around 200 bp, around 400 bp, and around 700 bp in the control group can be seen respectively around 100 bp, around 200 bp, around 400 bp, and around 700 bp in the result corresponding to the method of the invention. By contrast, the profile in FIG. 4C, which corresponds to the ThruPLEX method, not only fails to show the shape of the original cfDNA profile precisely (meaning the ThruPLEX method failed to amplify the cfDNA in the saliva sample with precision), but also shows an artifact at about 34 bp (as indicated by the small arrow A in FIG. 4C). It is obvious that the cfDNA amplified by the ThruPLEX method does not show the profile of the original cfDNA precisely. Therefore, the experimental results of the saliva samples have proved that, without having to perform such conventional PCR steps as purification with a spin column before the PCR, the method of the invention can produce satisfactory nucleic acid amplification results that are unachievable with the commercially available DNA amplification kit.

FIG. 5A shows the experimental result of the urine control group, or more particularly the profile of 150 ng of cfDNA in a purified yet unamplified urine sample. FIG. 5B shows the profile of 150 ng of cfDNA taken from PCR products generated from 10 μL of unpurified urine sample by PCR amplification performed according to the method of the present invention. FIG. 5C shows the profile of 150 ng of cfDNA taken from PCR products generated from 10 μL of unpurified urine sample by PCR amplification performed according to the ThruPLEX method. The peaks (indicated by the arrows as 20 LM and 5000 UM respectively) at the two end points of each profile in FIG. 5A, FIG. 5B, and FIG. 5C represent 20 bp and 5000 bp size markers, respectively.

Compared with the original profile in FIG. 5A, the profile in FIG. 5B, which was obtained after amplification according to the method of the present invention, shows with precision the shape of the original profile of the cfDNA in the control urine sample. More specifically, the data peaks around 100 bp and around 700 bp in the control group can be seen respectively around 100 bp and around 700 bp in the result corresponding to the method of the invention. By contrast, the profile in FIG. 5C, which corresponds to the ThruPLEX method, not only fails to show the shape of the original cfDNA profile precisely (meaning the ThruPLEX method failed to amplify the cfDNA in the urine sample with precision), but also shows an artifact at about 34 bp (as indicated by the small arrow A in FIG. 5C). It is obvious that the cfDNA amplified by the ThruPLEX method does not show the profile of the original cfDNA precisely. Therefore, the experimental results of the urine samples have proved that, without having to perform such conventional PCR steps as purification with a spin column or magnetic beads before the PCR, the method of the invention can produce satisfactory nucleic acid amplification results that are unachievable with the commercially available DNA amplification kit.

According to the above, the present invention dispenses with the nucleic acid purification process conventionally required before PCR and carries out PCR directly by virtue of the ligation efficiency of double-stranded homogeneous adapters, i.e., double-stranded adapters having a single type of sequence. Thus, all the nucleic acid fragments in a sample can undergo PCR and subsequent reactions in the same test tube, without any purification steps performed in advance, and this prevents loss of the nucleic acid fragments effectively. Moreover, the invention can amplify specific gene fragments of various sizes in a nucleic acid sample while maintaining high sensitivity and accuracy, thereby reducing the expenses and operation time of experiments effectively, the objective being to achieve economy in experimentation. In addition, the invention can provide even higher sensitivity and accuracy by using a test tube equipped with the filter disclosed herein.

The above is the detailed description of the present invention. However, the above is merely the preferred embodiment of the present invention and cannot be the limitation to the implement scope of the invention, which means the variation and modification according to the present invention may still fall into the scope of the present invention.

1. A method for amplifying free nucleic acids directly from an unpurified sample, wherein the unpurified sample comprises nucleic acid fragments, the method comprising the sequential steps of:
(a) dilution: mixing the unpurified sample with a buffer thoroughly to form a mixed solution;
(b) protein denaturation: adding the mixed solution into a test tube, heating the mixed solution in the test tube to denature proteins in the mixed solution, and then cooling the mixed solution to room temperature;
(c) end modification of nucleic acid molecules: subjecting the nucleic acid fragments in the mixed solution to a processing reaction required for an adapter-dependent polymerase chain reaction (PCR);
(d) adapter ligation: performing a ligation reaction between the processed nucleic acid fragments and double-stranded adapters having a single type of sequence, defined as double-stranded homogeneous adapters, in order to form nucleic acid fragments each ligated at each of two ends thereof with one said double-stranded homogeneous adapter, wherein each said double-stranded homogeneous adapter is a complementary double-stranded nucleic acid (dsNA) fragment with one strand being an oligonucleotide carrying a 5′-phosphate group and the other strand being an oligonucleotide carrying a thymine (T) or uracil (U); and
(e) PCR: adding components required for performing the adapter-dependent PCR on the ligated nucleic acid fragments, and then performing the adapter-dependent PCR on the ligated nucleic acid fragments in the mixed solution.

2. The method of claim 1, wherein the nucleic acid fragments in the unpurified sample are dsNA fragments.

3. The method of claim 1, wherein the unpurified sample is in an amount of 0.1 to 100 μL.

4. The method of claim 1, wherein the steps (b) to (e) are carried out in the same test tube.

5. The method of claim 4, wherein the test tube is provided therein with a filter, and the step (b) comprises adding the mixed solution onto the filter in order for the mixed solution to pass through the filter and then flow to a bottom of the test tube.

6. The method of claim 1, wherein the processing reaction in the step (c) is either a 3′-A-tailing reaction performed on the nucleic acid fragments in the mixed solution in order for the nucleic acid fragments to form nucleic acid fragments each having a 3′-A sticky end, or end modification performed on the nucleic acid fragments in the mixed solution in order for the nucleic acid fragments in the mixed solution to form nucleic acid fragments each having a blunt end, wherein the blunt ends are formed by repairing the nucleic acid fragments in the mixed solution or cleaving the nucleic acid fragments in the mixed solution with an enzyme.

7. The method of claim 1, wherein the adapter-dependent PCR in the step (e) is performed by using the nucleic acid fragments ligated with the double-stranded homogeneous adapters as templates, and by using a single type of bidirectional primer corresponding to the double-stranded homogeneous adapters.

8. The method of claim 1, wherein the adapter-dependent PCR is a digital PCR (dPCR).

9. The method of claim 8, further comprising the step (f) of purifying a product of the adapter-dependent PCR.

10. The method of claim 1, further comprising the step (g) of analyzing a product of the adapter-dependent PCR by sequencing or other diagnostic methods.
